(19) United States
(12) Patent Application Publication
(10) Pub. No.: US 2024/0238489 A1
(43) Pub. Date: Jul. 18, 2024

(54) Wearable and hidden breast pump

(71) Applicant: Yongqiang Zhang, Fuyang (CN)

(72) Inventor: Yongqiang Zhang, Fuyang (CN)

(21) Appl. No.: 18/181,905

(22) Filed: Mar. 10, 2023

(30) Foreign Application Priority Data
Jan. 12, 2023 (CN) 202320134540.3

(51) Int. Cl. A61M 1/06; A61M 1/00

(52) U.S. Cl. CPC A61M 1/066; A61M 1/067; A61M 1/06935; A61M 1/802; A61M 2205/3344

(57) ABSTRACT

A wearable and hidden breast pump includes a shell, a flange, a negative pressure chamber, a negative pressure pump, a milk container and a solenoid valve. The flange is arranged on a front surface of the shell; the negative pressure chamber is arranged in the shell and connected with the flange to form an lactation chamber; the negative pressure pump and the solenoid valve are arranged in the shell, the negative pressure pump, the solenoid valve and the negative pressure chamber are communicated; the negative pressure pump intermittently extracts air from the negative pressure chamber, and the solenoid valve intermittently inflates air into the negative pressure chamber, thus controlling an air amount in said negative pressure chamber, thereby controlling the lactation chamber to form a negative pressure or a positive pressure; the milk container is arranged under the shell and communicated with the lactation chamber.

### FIELD OF THE INVENTION

The present invention relates to breast pump, more particularly, to a wearable and hidden breast pump.

### BACKGROUND OF THE INVENTION

When mothers are unable to stay with infants or their physical conditions are not suitable for feeding the infants, they usually extract milk from their breasts with breast pumps to store in milk bottles to feed the infants.

Currently, the general breast pump in the market has a cup attached to the breast, a motor, and a container for storing milk. This kind of breast pump is large, therefore it is inconvenient to carry, and it cannot be hidden in the mother's clothes when it is used and is not suitable for use in public places. The mothers must find a hidden private space, and for working mothers, it is especially inconvenient, thus it reduces the user's experience of use.

Therefore, people develop a wearable breast pump, which can be hidden in underwear, it solves the deficiencies that the currently breast pump is large and inconvenient to carry, and not suitable for use in public places. Chinese patent CN216571014U discloses a wearable breast pump, which includes a motor, a cylinder, a milk storage cup, a horn cover, and a valve, the motor is connected with the cylinder, the cylinder is connected with the milk storage cup, the milk storage cup is connected with the horn cover, the valve is connected with the horn cover. The motor is used to control the cylinder to produce negative pressure at the horn cover so as to cause the breast to express the milk. The valve is a one-way valve, which is closed when the cylinder is in negative pressure state, and is open and connects the horn cover and the milk storage cup when the cylinder recovers its shape so as to drain the milk into the milk storage cup.

However, in order to ensure sufficient suction, the current wearable breast pump usually cannot reduce the volume of the main machine, the cylinder and the air chamber, which makes the wearable breast pump large in volume, heavy in weight, and the suction of the wearable breast pump cannot be guaranteed. The small suction leads to an low suction efficiency, which also leads to bad use experience from the users.

### SUMMARY OF THE INVENTION

To overcome the shortcomings in the prior art, the present invention provides a wearable and hidden breast pump, which overcomes the shortcomings in the current wearable breast pump, for example being large in volume, heavy, and low suction efficiency, etc. By an ingenious and reasonable structural design, the wearable and hidden breast pump has a small negative pressure chamber, an adjustable negative pressure, a high suction efficiency, and the users will have a good experience if they use it.

A wearable and hidden breast pump includes a shell, a flange, a negative pressure chamber, a negative pressure pump, a milk container, and a solenoid valve. The flange is arranged on a front surface of the shell; the negative pressure chamber is arranged in the shell and connected with the flange to form a lactation chamber; the negative pressure pump and the solenoid valve are arranged in the shell, the negative pressure pump, the solenoid valve and the negative pressure chamber are communicated, the negative pressure pump intermittently extracts air from the negative pressure chamber and alternatively the solenoid valve intermittently inflates air into the negative pressure chamber, thus controlling an air volume in the negative pressure chamber, thereby controlling the lactation chamber in a negative pressure state or a positive pressure state; the milk container is arranged under the shell and communicated with the lactation chamber.

The negative pressure chamber is provided with a through hole, one end of a tee valve is communicated with the negative pressure chamber through the through hole, another two ends of the tee valve are respectively communicated with the negative pressure pump and the solenoid valve; extracting air from the negative pressure chamber and inflating air into the negative pressure chamber are alternatively, thereby controlling the air amount in therein.

The negative pressure chamber includes a front cover, a rear cover, and an anti-counter-flow membrane. The anti-counter-flow membrane is made of flexible and elastic material, such as silicone, disposed between the front cover and the rear cover and connected with the front cover and the rear cover in a sealed way. A sealed chamber is disposed between the anti-counter-flow membrane and the rear cover, the tee valve is communicated with the sealed chamber, and the lactation chamber is disposed between the anti-counter-flow membrane and the flange.

The front cover has a guide cylinder, one end thereof is laterally provided with a front cover corrugated part, and a periphery thereof is provided with a front cover connecting part. The rear cover has a cover body, a rear cover connecting part arranged on a periphery of the cover body, and an inner ring arranged on the cover body. The rear cover connecting part is connected with the front cover connecting part, and a gap is formed between the inner ring and the front cover connecting part. The anti-counter-flow membrane has a deforming part, a corrugated part, and a sealing part. The deforming part is inserted into the guide cylinder, the corrugated part fits the front cover corrugated part, and the sealing part is inserted into the gap, fitting the front cover connecting part and the inner ring, thereby connecting them in a sealed way. The sealed chamber is formed among the cover body, the inner ring, and the anti-counter-flow membrane.

The front cover or the rear cover is provided with the through hole, a sealing ring seals the through hole, thereby sealing the sealed chamber.

The shell is provided with a mounting base, the front cover is matched with the mounting base, thereby conveniently installing the front cover.

A bottom of the guide cylinder is provided with a flow-guiding bore, the milk container has an intake facing the flow-guiding bore, thereby the milk can be gathered in the milk container through the flow-guiding bore.

The bottom of the guide cylinder is provided with a convex ring surrounding the flow-guiding bore, and the one-way valve in silicone is arranged at the convex ring and extends into the milk container.

The one-way valve in silicone has a mounting ring and a V-shaped pipe arranged on the mounting ring, the mounting ring is matched with the convex ring and connected with it in a sealed way, and an end of the V-shaped pipe is open.

The shell includes comprises a front shell, a rear hell, and a decorative framework connecting the front shell and the rear hell to form a cavity, where the negative pressure chamber and the negative pressure pump are mounted. The front shell and a front wall of the milk container are matched to form a cavity in a trumpet shape. A middle part of the front shell forms a flow-guiding outer cylinder; the flange matches the front shell and the front wall of the milk container in shape and is mounted in the cavity in trumpet shape; the flange is made of a flexible and elastic material, such as silica gel, and closely fits the front shell and the front wall of the milk container; a middle part of the flange forms a flow-guiding inner cylinder; and the guiding inner cylinder is connected to the guiding cylinder in a sealed way so that the lactation chamber is enclosed among the flange, the guide cylinder and the anti-counter-flow membrane.

In the cavity, one side of the mounting base is provided with the negative pressure pump, and the other side opposite thereto is provided with the solenoid valve and a battery.

The cavity is provided with a PCB circuit board, which is electrically connected to the negative pressure pump and the solenoid valve; The shell is provided with a power supply interface for charging the battery via an external power supply, and the battery is electrically connected to the PCB circuit board to supply power to the negative pressure pump and the solenoid valve.

The milk container includes a front cover, a rear cover, and a sealing strip; the front cover is disposed below the front shell, and a shape thereof matches the front shell; the rear cover is located below the rear shell, and the shape thereof matches the rear shell; the front cover and the rear cover are respectively provided a nut and a matching groove at a joint thereof, and the sealing strip is accommodated between the nut and the matching groove, thereby connecting them in a sealed way.

The intake and two mounting plates are disposed on a top surface of the rear cover; the two mounting plates are respectively provided with a mounting groove, and the two mounting plates are respectively provided at two sides of the intake; two insertion holes are disposed on a top surface of the rear shell; the mounting base is provided with two insertion grooves at a position corresponding to the two insertion holes; two elastic parts are respectively disposed on a groove wall of the two insertion grooves; the two mounting plates are respectively penetrated through the two insertion holes and are respectively inserted into the two insertion grooves, and the elastic parts are elastically inserted into the mounting grooves respectively so that the milk container is mounted on the rear shell.

The wearable and hidden breast pump in the present invention has a reasonable structure design, a small volume and a lightweight, and is convenient for hidden wearing, and is comfortable and convenient to wear.

### BRIEF DESCRIPTION OF THE DRAWINGS

In order to more clearly illustrate the technical schemes in the embodiments in the present invention, the drawings needed in the embodiments or the prior technical description are briefly introduced below. Obviously, the drawings described below are only some embodiments in the present invention, and for those skilled in the art, additional drawings may be obtained according to the drawings below without creative work.

FIG. 1 is a perspective view of the wearable and hidden breast pump according to the present invention.

FIG. 2 is an exploded view of the wearable and hidden breast pump according to the present invention.

FIG. 3 is the other exploded view of the wearable and hidden breast pump according to the present invention.

FIG. 4 is another exploded view of the wearable and hidden breast pump according to the present invention.

FIG. 5 is another exploded view of the wearable and hidden breast pump according to the present invention.

FIG. 6 is a perspective view of the negative pressure chamber, the negative pressure pump and the solenoid valves according to the present invention.

FIG. 7 is a front view of the wearable and hidden breast pump according to the present invention.

FIG. 8 is a sectional view along line A-A in FIG. 7.

FIG. 9 is a sectional view along line B-B in FIG. 7.

FIG. 10 is another exploded view of the wearable and hidden breast pump according to the present invention.

FIG. 11 is is another exploded view of the wearable and hidden breast pump according to the present invention.

FIG. 12 is a top view of the wearable and hidden breast pump according to the present invention.

FIG. 13 is a sectional view along line C-C in FIG. 11.

### DETAILED DESCRIPTION OF ILLUSTRATED EMBODIMENTS

To make the objectives, technical solutions, and technical effects of the present invention clearer and more comprehensible, the following further describes the present invention with reference to embodiments. It should be understood that the embodiments described herein are only used to explain the present invention, but not used to limit the present invention.

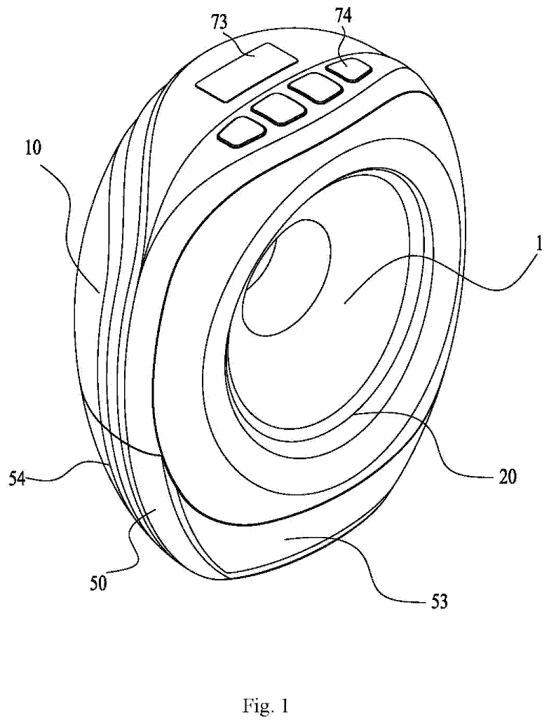

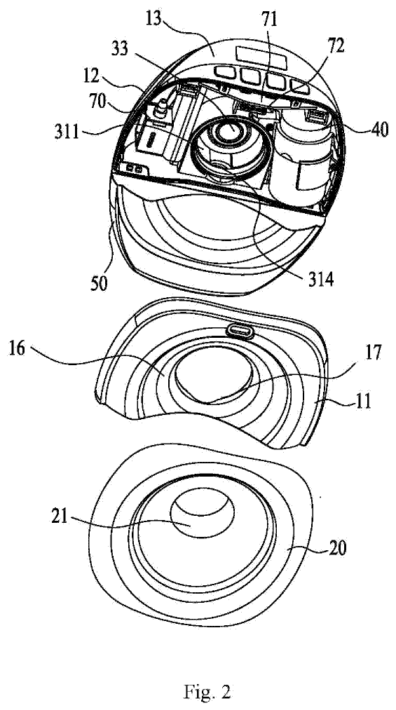

Referring to FIG. 1-5, a wearable and hidden breast pump includes a shell 10, a flange 20, a negative pressure chamber 30, a negative pressure pump 40, a milk container 50, and a solenoid valve 60. The flange 20 is arranged on a front surface of the shell 10 used for fitting the breasts of the mother. The negative pressure chamber 30 is arranged in the shell 10 and connected with the flange 20 to form a lactation chamber 1. The negative pressure pump 40 and the solenoid valve 60 are arranged in the shell 10. The negative pressure pump 40, the solenoid valve 60, and the negative pressure chamber 30 are communicated, the negative pressure pump 40 intermittently extracts air from the negative pressure chamber 30, alternatively, the solenoid valve 60 intermittently inflates air into the negative pressure chamber 30, thus controlling the air volume in the negative pressure chamber 30, thereby controlling the lactation chamber 1 in a negative pressure state or a positive pressure state, thereby simulating the sucking effect of an infant and promoting the milk secretion in a mild and highly efficient way; the milk container 50 is arranged under the shell 10 and communicated with the lactation chamber 1.

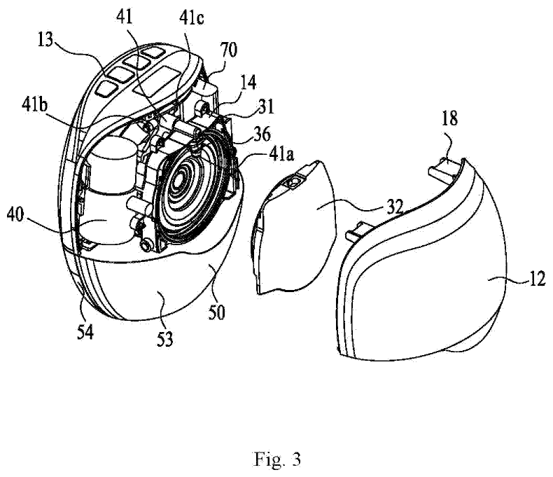

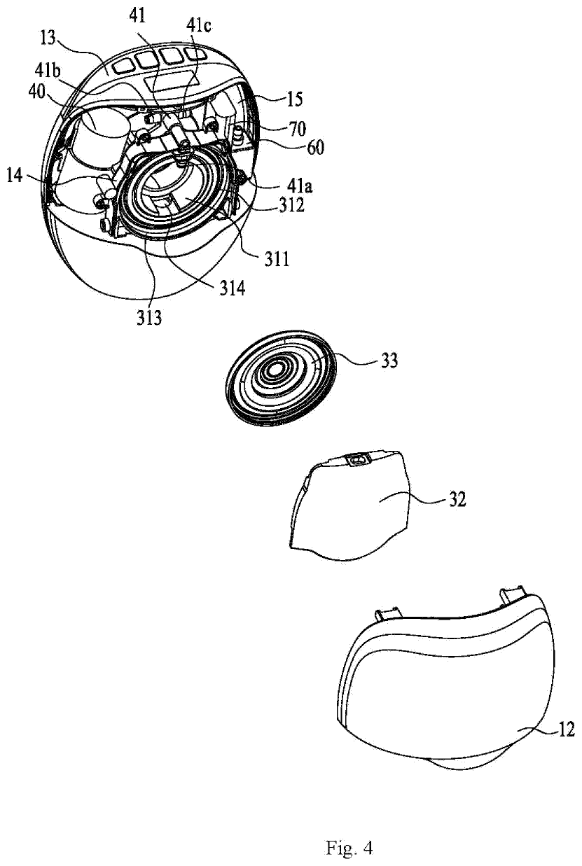

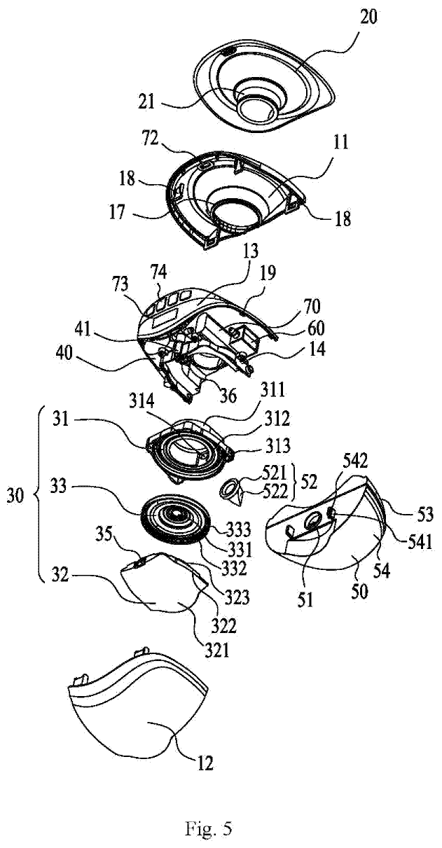

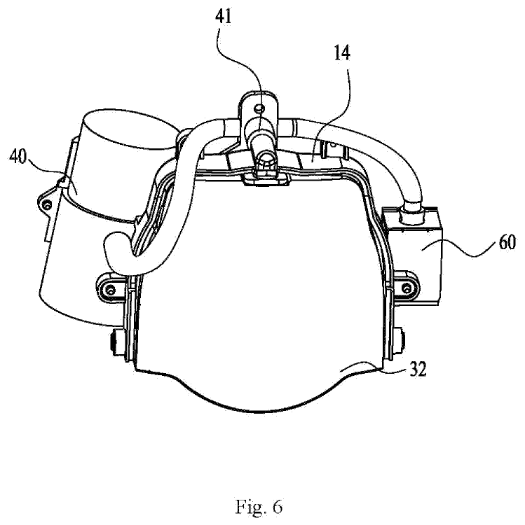

Specifically, referring to FIG. 3, FIG. 4 and FIG. 5, the negative pressure chamber 30 is provided with a through hole 35, one end 41a of a tee valve 41 is communicated with the negative pressure chamber 30 through the through hole 35, another two ends 41b and 41c of the tee valve 41 are respectively communicated with the negative pressure pump 40 and the solenoid valve 60; extracting air from the negative pressure chamber 30 and inflating air into the negative pressure chamber 30 are alternatively operated, thereby controlling the air amount in the negative pressure chamber 30. To clearly illustrate the structure of each component, a few of the figures in the present invention do not show a gas pipe in communication with the tee valve 41, the negative pressure pump 40, and the solenoid. The gas pipe is shown in FIG. 6. As shown in FIG. 6, there are two gas pipes, one of which is in communication with the tee valve 41 and the negative pressure pump 40, and the other of which is in communication with the tee valve 41 and the solenoid valve 60. It should be understood that structures and principles of the negative pump and the solenoid valve 60 are known technologies to persons skilled in the art, and are not described in detail in this present invention.

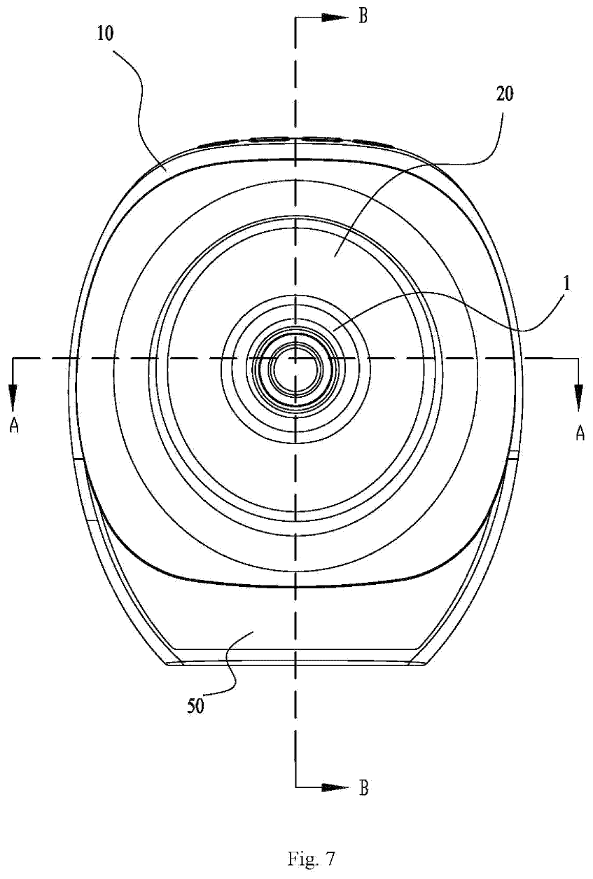

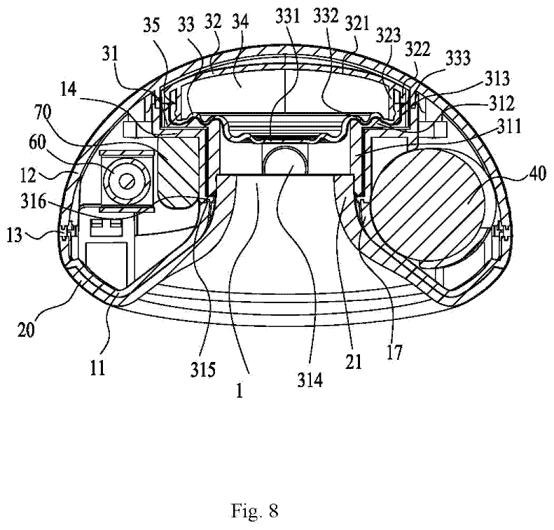

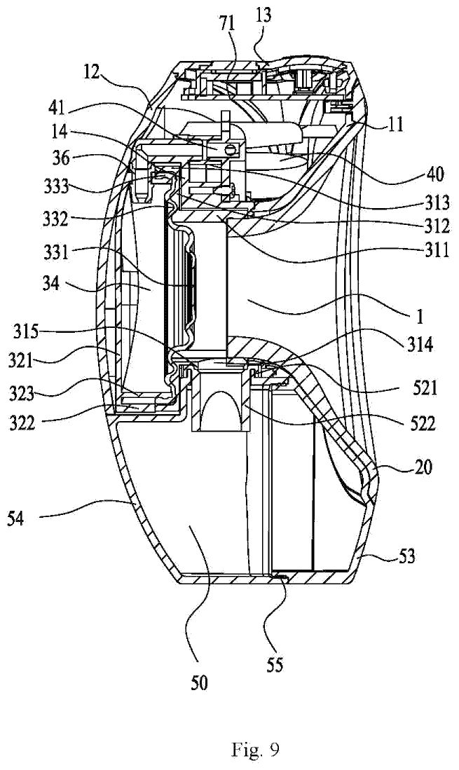

Specifically, referring to FIG. 5, FIG. 8 and FIG. 9, the negative pressure chamber 30 includes a front cover 31, a rear cover 32, and an anti-counter-flow membrane 33. The anti-counter-flow membrane 33 is made of flexible and elastic material, such as silicone, disposed between the front cover 31 and the rear cover 32 and connected with the front cover 31 and the rear cover 32 in a sealed way. A sealed chamber 34 is formed between the anti-counter-flow membrane 33 and the rear cover 32, the tee valve 41 is communicated with the sealed chamber 34, and the lactation chamber 1 is disposed between the anti-counter-flow membrane 33 and the flange 20. When the wearable and hidden breast pump is used, the flange 20 closely fits the breast of the mother. The negative pressure pump 40 extracts air from the sealed chamber 34 through the tee valve 41 to form negative pressure in it, at this time, the solenoid valve 60 is closed, pressure in the lactation chamber 1 is higher than that in the sealed chamber 34, so that the anti-counter-flow membrane 33 is deformed into the sealed chamber 34, thereby reducing the volume of the sealed chamber 34, expanding the volume of the lactation chamber 1, and forming a negative pressure in the lactation chamber 1. Then, the negative pressure pump 40 stops extracting air, the solenoid valve 60 is operated to inflate air into the sealed chamber 34. The anti-counter-flow membrane 33 is reset to deform in the direction of the lactation chamber 1, the volume of the sealed chamber 34 is increased, the volume of the lactation chamber 1 is reduced, and a positive pressure is formed in the lactation chamber 1.

That, the negative pressure pump 40 extracts air from the sealed chamber 34 and the solenoid valve 60 inflates air into the sealed chamber 34, is operated alternatively, so that the sealed chamber 34 can simulate the sucking effect of the infant and promote the breast of the mother to secrete milk. Since the negative pressure pump 40 and the solenoid valve 60 do not directly work on the lactation chamber 1 to form a negative pressure or a positive pressure, they directly work on the sealed chamber 34 to cause the anti-counter-flow membrane 33 to deform, thereby controlling the lactation chamber 1 to form the negative pressure or positive pressure, the pressure is gentle so that the breasts feel comfortable, it does not cause pain, and the mother has a good experience. The negative pressure chamber 30 has a simple structure, a lightweight, and a small volume, so that the whole wearable and hidden breast pump has a lightweight, a small volume, and is easy to wear.

Referring to FIG. 5, FIG. 8 and FIG. 9, the front cover 31 has a guiding cylinder 311, one end of the guiding cylinder 311 is laterally provided with a front cover corrugated part 312, and a periphery of the front cover corrugated part 312 is provided with a front cover connecting part 313. The rear cover 32 has a cover body 321 and a rear cover connecting part 322 arranged on a periphery of the cover body 321, and an inner ring 323 arranged on the cover body 321. The rear cover connecting part 322 is connected with the front cover connecting part 313, and a gap 324 is formed between the inner ring 323 and the front cover connecting part 313. The anti-counter-flow membrane 33 has a deforming part 331, a corrugated part 332, and a sealing part 333. The deforming part 331 is inserted into the guiding cylinder 311, the corrugated part 332 fits the front cover corrugated part 312, the sealing part 333 is inserted into the gap 324, fitting the front cover connecting part 313 and the inner ring 323, thereby connecting them in a sealed way. The sealed chamber 34 is formed among the cover body 321, the inner ring 323, and the anti-counter-flow membrane 33. In order to facilitate adjustment of the volume of the sealed chamber 34 by means of air extraction or inflation, the anti-counter-flow membrane 33 must be easy to deform and easy to reset. Therefore, the anti-counter-flow membrane 33 is made of a flexible and elastic material, such as silicon glue, and has a thickness of 0.2-3 cm. The thickness of the deforming part 331 is preferably 0.2-0.5 cm.

Referring to FIG. 3, FIG. 5 and FIG. 8, the negative pressure pump 40 is in communication with the sealed chamber 34. Specifically, the through hole 35 is provided at the front cover 31 or the rear cover 32, or the junction of the front cover 31 and the rear cover 32, and one path 41a of the tee valve 41 is in communication with the sealed chamber 34 through the through hole 35. A sealing ring 36 seals the through hole 35, thereby sealing the sealed chamber 34.

When the negative pressure pump 40 extracts air from the sealed chamber 34, the deforming part 331 of the anti-counter-flow membrane 33 is deformed in the direction of the sealed chamber 3 so as to reduce the volume of the sealed chamber 34, expand the volume of the lactation chamber 1, thereby forming a negative pressure in the lactation chamber 1, and stimulating the negative pressure effect on the breast formed by a suction action of infant and secret milk. When the solenoid valve 60 inflates to the sealed chamber 34, the deforming part 331 of the anti-counter-flow membrane 33 is deformed in the direction of the lactation chamber 1 so as to expand the volume of the sealed chamber 34and reduce the volume of the lactation chamber 1, thereby forming a positive pressure in the lactation chamber 1, the mill can flow out.

Referring to FIG. 4 and FIG. 5, the shell 10 is provided with a mounting base 14, the front cover 31 is matched with the mounting base 14, thereby conveniently installing the front cover 31. Specifically, the front cover 31 is mounted on the mounting base 14 through clamping, screw connection, etc., and the mounting is simple and convenient.

The milk container 50 is disposed on the bottom of the guiding cylinder 311. A bottom of the guiding cylinder 311 is provided with a flow-guiding bore 314, the milk container 50 has an intake 51 facing the flow-guiding bore 314, thereby the milk can be gathered in the milk container 50 through the flow-guiding bore 314. The milk container 50 is arranged below the negative chamber, the milk can naturally flow into the milk container 50 under the action of gravity, thereby facilitating the collection of the milk, avoiding waste, and having a reasonable structure design.

Referring to FIG. 5 and FIG. 9, the bottom of the guiding cylinder 311 is provided with a convex ring 315 surrounding the flow-guiding bore 314, the one-way valve 52 in silicone is arranged at the convex ring 315 and extends into the milk container 50. The milk flows into the milk container 50 through the flow-guiding bore 314 and the one-way valve 52 in silicone. Specifically, the one-way valve 52 in silicone has a mounting ring 521 and a V-shaped pipe 522 arranged on the mounting ring 521, the mounting ring 521 is matched with the convex ring 315 and connected with it in a sealed way, an end of the V-shaped pipe 522 is open. When the pressure in the lactation chamber 1 is negative, the tube wall of the V-shaped pipe 522 is fitted closely the end, thereby facilitating the formation of negative pressure in the lactation chamber 1. When the pressure in the lactation chamber 1 is positive, the end of the V-shaped pipe 522 is opened, so that milk can flow from the lactation chamber 1 into the milk container 50.

Referring to FIG. 5 and FIG. 9, the shell 10 includes comprises a front shell 11, a rear hell 12, and a decorative framework 13 connecting the front shell 11 and the rear hell 12 to form a cavity 15, where the negative pressure chamber 30 and the negative pressure pump 40 are mounted. The milk container 50 is disposed below the shell 10 and matches the shape of the shell 10 to form an oval milk suction device. Specifically, the front shell 11 and a front wall of the milk container 50 are matched in shape to form a cavity in trumpet shape 16. A middle part of the front shell 11 forms a flow-guiding outer cylinder 17. The flow-guiding outer cylinder 17 and the mounting base 14 are respectively provided with a convex ring 315 and a groove 316, and the convex ring 315 and the groove 316 are clamped to connect the flow-guiding outer cylinder 17 and the mounting base 14.

The flange 20 matches the front shell 11 and the front wall of the milk container 50 in shape and is mounted in the cavity in a trumpet shape 16. The flange 20 is made of a flexible and elastic material, such as silica gel, and closely fits the front shell 11 and the front wall of the milk container 50. A middle part of the flange 20 forms a flow guiding inner cylinder 21. And the flow guiding inner cylinder 21 passed through the mounting base 14 and is connected to the guiding cylinder 311 in a sealed way, so that the lactation chamber 1 is enclosed among the flange 20, the guiding cylinder 311 and the anti-counter-flow membrane 33. The mick secreted by the breast flows through the flange 20, and the flow guiding inner cylinder 21, into the guiding cylinder 311, and then through the flow-guiding bore 314 and the silicone one-way valve 52 into the milk container 50. The flow process is natural and smooth, and the milk will not accumulate and remain in the lactation chamber 1. The flange 20 adopts a bionic design that simulates the shape of the breast, and adopts a skin-friendly flexible silicone material, which is comfortable to wear and easy to disassemble and clean.

The decorative framework 13 and the front shell 11, as well as the decorative framework 13 and the rear shell 12 are respectively provided with a matched snap ring 18 and a snap groove 19, which are clamped and connected with each other.

In the cavity 15, one side of the mounting base 14 is provided with the negative pressure pump 40, and the other side opposite thereto is provided with the solenoid valve 60 and a battery 70. The negative pressure pump 40, the solenoid valve 60 and the battery 70 are respectively disposed two sides of the mounting base 14 to make full use of the cavity 15. The structure design is reasonable, making the structure of the breast pump small and convenient for wearing.

The cavity 15 is provided with a PCB circuit board 71, which is electrically connected to the negative pressure pump 40 and the solenoid valve 60. The shell 10 is provided with a power supply interface 72 for charging the battery 70 via an external power supply, and the battery 70 is electrically connected to the PCB circuit board 71 and the negative pressure pump 40 to supply power to them.

The shell 10 is provided with a display screen 73 and an operation key 74, which is convenient for controlling the operation of the negative pressure pump 40 and setting the working parameters.

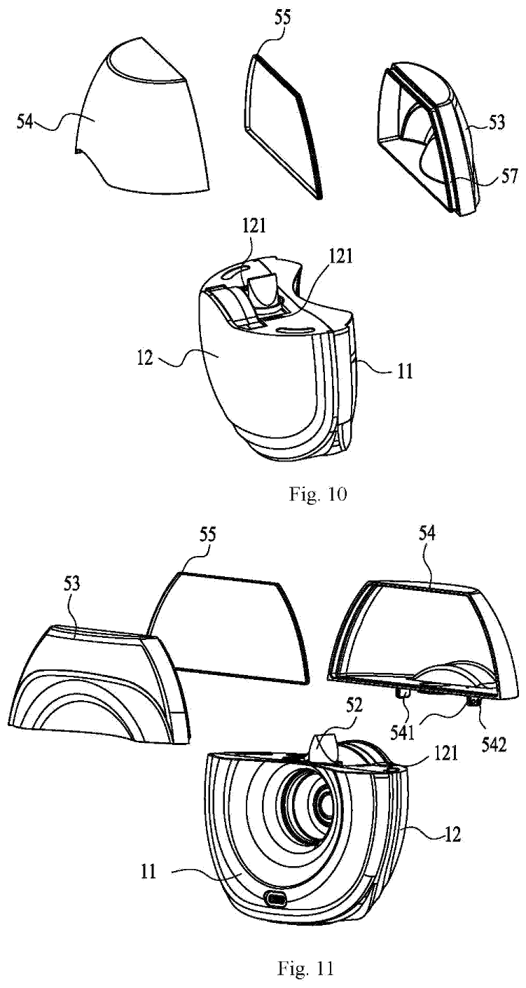

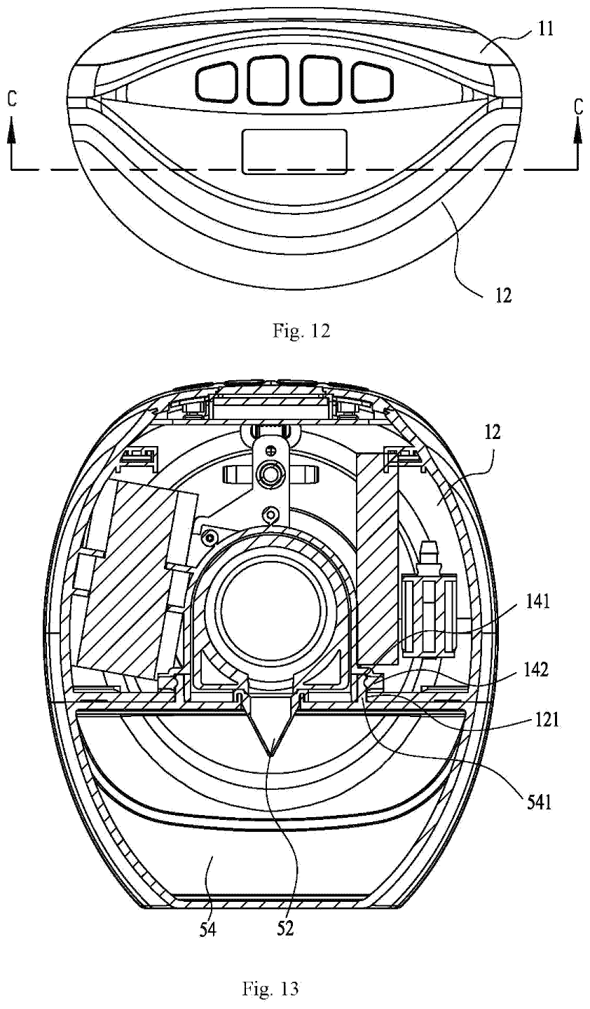

Referring to FIG. 10, FIG. 11, FIG. 12 and FIG. 13, the milk container 50 includes a container front cover 51, a container rear cover 52, and a sealing strip 53. The container front cover 51 is disposed below the front shell 11, and a shape thereof matches the front shell 11. Specifically, the front wall of the container front cover 51 and the front wall of the front shell 11 are respectively depressed to form the cavity in a tramped shape 16 together. The container rear cover 52 is located below the rear shell 12, and the shape thereof matches the rear shell 12 to form an arc surface. The container front cover 51 and the container rear cover 52 are respectively provided a nut 56 and a matching groove 57 at a joint thereof, and the sealing strip 53 is accommodated between the nut 56 and the matching groove 57, thereby connecting them in a sealed way.

The intake 51 and two mounting plates 541 are disposed on a top surface of the container rear cover 54; the two mounting plates 541 are respectively provided with a mounting groove 542, and the two mounting plates 541 are respectively provided at two sides of the intake 51; two insertion holes 121 are disposed on a top surface of the rear shell 12; the mounting base 14 is provided with two insertion grooves 141 at a position corresponding to the two insertion holes 121; two elastic parts 142 are respectively disposed on a groove wall of the two insertion grooves 141; the two mounting plates 541 are respectively penetrated through the two insertion holes 121 and are respectively inserted into the two insertion grooves 141, and the elastic parts 142 are elastically inserted into the mounting grooves 542 respectively, so that the milk container 50 is mounted on the rear shell 12. When the milk container 50 needs to be disassembled, overcome the matching connection between the elastic parts 142 and the mounting grooves 542 and pull the milk container 50 out of the mounting grooves 542 and the insertion holes. Through the elastic clamping installation structure, the installation and disassembly of the milk container 50 are realized, and the installation is stable and the disassembly is convenient.

After the milk container 50 is disassembly, pour out milk from the intake 51, disassembly the milk container 50, clean and disinfect the front cover 31, the rear cover 32 and the sealing strip of the milk container 50 for the next use.

The corners of the front cover 31 and the rear cover 32 of the milk container 50 are arc chambered, which is convenient for cleaning, prevents milk residue and has a beautiful shape.

The above contents are further detailed instructions to the invention in combination with the specific optimal embodiments, and it can not be concluded that the embodiments of the invention are limited to these instructions. For ordinary technicians in the technical field of the invention, without breaking away from the conception of the invention, the structure form can be flexible and changeable, and the series of products can be derived. Only a few simple deductions or replacements should be regard in the protection scope of the present invention.

What is claimed is:

1. A wearable and hidden breast pumpcomprising:
a shell;
a flange, arranged on a front surface of said shell;
a negative pressure chamber, arranged in said shell and connected with said flange to form a lactation chamber;
a milk container, arranged under said shell and communicated with said lactation chamber
a negative pressure pump, arranged in said shell; and
a solenoid valve, arranged in said shell; said negative pressure pump, said solenoid valve, and said negative pressure chamber are communicated; said negative pressure pump intermittently extracts air from said negative pressure chamber, and alternatively, said solenoid valve intermittently inflates air into said negative pressure chamber, thus controlling an air amount in said negative pressure chamber, thereby controlling said lactation chamber to form a negative pressure or a positive pressure.

2. The wearable and hidden breast pump according to claim 1wherein said negative pressure chamber is provided with a through hole, one end of a tee valve is communicated with said negative pressure chamber through said through hole, another two ends of said tee valve are respectively communicated with said negative pressure pump and said solenoid valve; said negative pressure pump extracting air from said negative pressure chamber and said solenoid valve inflating air into said negative pressure chamber are operated alternatively, thereby controlling said air amount therein.

3. The wearable and hidden breast pump according to claim 2wherein said negative pressure chamber including a front cover, a rear cover and a anti-counter-flow membrane; said anti-counter-flow membrane is made of flexible and elastic material, disposed between said front cover and said rear cover, and connected with said front cover and said rear cover in a sealed way; a sealed chamber is formed between said anti-counter-flow membrane and said rear cover, and said tee valve is communicated with said sealed chamber, and said lactation chamber is disposed between said anti-counter-flow membrane and said flange.

4. The wearable and hidden breast pump according to claim 3wherein said front cover has a guide cylinder, one end of said guide cylinder is laterally provided with a front cover corrugated part, and a periphery of said front cover corrugated part is provided with a front cover connecting part; said rear cover has a cover body, a rear cover connecting part arranged on a periphery of said cover body, and an inner ring arranged on said cover body; said rear cover connecting part is connected with said front cover connecting part, and a gap is formed between said inner ring and said front cover connecting part; said anti-counter-flow membrane has a deforming part, a corrugated part and a sealing part; said deforming part is inserted into said guide cylinder, said corrugated part fits said front cover corrugated part, and said sealing part is inserted into said gap, fitting said front cover connecting part and said inner ring, thereby connecting them in a sealed way; said sealed chamber is formed among said cover body, said inner ring, and said anti-counter-flow membrane.

5. The wearable and hidden breast pump according to claim 3wherein said front cover or said rear cover is provided with said through hole, a sealing ring seals said through hole, thereby sealing said sealed chamber.

6. The wearable and hidden breast pump according to claim 3wherein said shell is provided with a mounting base, said front cover is matched with said mounting base, thereby conveniently installing said front cover.

7. The wearable and hidden breast pump according to claim 4wherein a bottom of said guide cylinder is provided with a flow-guiding bore, said milk container has an intake facing said flow-guiding bore, thereby said milk can be gathered in said milk container through said flow-guiding bore.

8. The wearable and hidden breast pump according to claim 7wherein said bottom of said guide cylinder is provided with a convex ring surrounding said flow-guiding bore, and said one-way valve in silicone is arranged at said convex ring and extends into said milk container.

9. The wearable and hidden breast pump according to claim 8wherein said one-way valve in silicone has a mounting ring and a V-shaped pipe arranged on said mounting ring, said mounting ring is matched with said convex ring and connected with it in a sealed way, and an end of said V-shaped pipe is open.

10. The wearable and hidden breast pump according to claim 1wherein said shell including a front shell, a rear hell, and a decorative framework connecting said front shell and said rear hell to form a cavity, where said negative pressure chamber and said negative pressure pump are mounted; said front shell and a front wall of the milk container are matched to form a cavity in a trumpet shape, a middle part of said front shell forms a flow-guiding outer cylinder;
said flange matches said front shell and said front wall of said milk container in shape and is mounted in said cavity in trumpet shape; said flange is made of a flexible and elastic material and closely fits said front shell and said front wall of said milk container; a middle part of said flange forms a flow-guiding inner cylinder; and said guiding inner cylinder is connected to said guiding cylinder in a sealed way so that said lactation chamber is enclosed among said flange, said guide cylinder and said anti-counter-flow membrane.

11. The wearable and hidden breast pump according to claim 10wherein in said cavity, one side of said mounting base is provided with said negative pressure pump, and the other side opposite thereto is provided with said solenoid valve and a batterysaid battery supplied said said negative pressure pump and said solenoid valve.

12. The wearable and hidden breast pump according to claim 11wherein said cavity is provided with a PCB circuit board, which is electrically connected to said negative pressure pump and said solenoid valve; said shell is provided with a power supply interface for charging said battery via an external power supply, and said battery is electrically connected to said PCB circuit board to supply power to said negative pressure pump and said solenoid valve.

13. The wearable and hidden breast pump according to claim 10wherein said milk container including:
a front cover, disposed below said front shell, and a shape thereof matches said front shell;
a rear cover, located below said rear shell, and said shape thereof matches said rear shell; and said front cover and said rear cover are respectively provided a nut and a matching groove at a joint thereof; and
a sealing strip, accommodated between said nut and said matching groove, thereby connecting them in a sealed way.

14. The wearable and hidden breast pump according to claim 7wherein said intake and two mounting plates are disposed on a top surface of said rear cover; said two mounting plates are respectively provided with a mounting groove, and said two mounting plates are respectively provided at two sides of said intake; two insertion holes are disposed on a top surface of said rear shell; said mounting base is provided with two insertion grooves at a position corresponding to said two insertion holes; two elastic parts are respectively disposed on a groove wall of said two insertion grooves; said two mounting plates are respectively penetrated through said two insertion holes and are respectively inserted into said two insertion grooves, and said elastic parts are elastically inserted into said mounting grooves respectively so that said milk container is mounted on said rear shell.
